SEQUENCE LISTING (6 sequences)

SEQ ID NO: 1 - DNA, 4073 bp, Artificial Sequence
  CDLV1
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaacaaagcttgccttgagtgcttcaagtagtgtgtgcccgtctgttgtgtgactctggtaactagagatccctcagacccttttagtcagtgtggaaaatctctagcagttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 2 - DNA, 3750 bp, Artificial Sequence
  CDLV2
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaacaaagcttgccttgagtgcttcaagtagtgtgtgcccgtctgttgtgtgactctggtaactagagatccctcagacccttttagtcagtgtggaaaatctctagcagttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 3 - DNA, 3969 bp, Artificial Sequence
  CDLV3
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaacaaagcttgccttgagtgcttcaagtagtgtgtgcccgtctgttgtgtgactctggtaactagagatccctcagacccttttagtcagtgtggaaaatctctagcagttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 4 - DNA, 4174 bp, Artificial Sequence
  CDLV4
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaacaaagcttgccttgagtgcttcaagtagtgtgtgcccgtctgttgtgtgactctggtaactagagatccctcagacccttttagtcagtgtggaaaatctctagcagttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 5 - DNA, 3754 bp, Artificial Sequence
  CDLV5
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaacaaagcttgccttgagtgcttcaagtagtgtgtgcccgtctgttgtgtgactctggtaactagagatccctcagacccttttagtcagtgtggaaaatctctagcagttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgatccagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 6 - DNA, 3519 bp, Artificial Sequence
  CDLV6
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacgaagtagtgtgtgcccgtctgttgtgtgactctggtaactagagatccctcagacccttttagtcagtgtggaaaatctctagcagttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgatccagacatgataagatacattgatgagtttggacaaaccacaactagaatgcagtgaaaaaaatgctttatttgtgaaatttgtgatgctattgctttatttgtaaccattataagctgcaataaacaagtt

(19) United States
(12) Patent Application Publication
(10) Pub. No.: US 2024/0041957 A1
(43) Pub. Date: Feb. 8, 2024

(54) RETROVIRAL VECTORS

(71) Applicant: UCL Business LTD, London (GB)

(72) Inventors: John Counsell, London (GB); Guillaume DE BRABANDERE, London (GB)

(21) Appl. No.: 18/255,444

(22) PCT Filed: Dec. 3, 2021
(86) PCT No.: PCT/GB2021/053169

§ 371 Date: Jun. 1, 2023

(30) Foreign Application Priority Data
Dec. 3, 2020 (GB) 2019084.9

(51) Int. Cl. A61K 35/76; C12N 15/86

(52) U.S. Cl. CPC A61K 35/76; C12N 15/86; C12N 2740/16043; C12N 2830/48; C12N 2740/16032

(57) ABSTRACT

There is provided a retroviral RNA vector comprising a 5′ cap, a transgene, a 3′ long terminal repeat (LTR) and an RNA packaging sequence, wherein translation of the transgene is initiated at the 5′ end of the transgene in a cap-dependent manner, and wherein the 3′ LTR and the RNA packaging sequence are located 3′ of the transgene. Also provided is a nucleotide sequence encoding a vector genome. In addition, there is provided a host cell, a virion and a pharmaceutical composition comprising the vector or nucleotide sequence, and the use of the vector in delivering a transgene to a cell or subject.

### FIELD OF THE INVENTION

The present invention relates to a retroviral vector which has been engineered such that the RNA vector genome is directly expressed and translated on target cell entry without reverse transcription or integration into the target cell genome. This retroviral vector results in improved translation of the RNA vector genome compared to previous technologies.

### BACKGROUND TO THE INVENTION

Lentiviral (LV) vectors based on Human Immunodeficiency Virus Type I (HIV-1) have been developed to deliver genetic material to a broad range of cell-types. Integration-proficient LV (IPLV) vectors are the conventional form of LV technology, which permanently deliver DNA sequences to patient cells, with the majority of delivered proviruses integrating into the transduced cell genome. But these integration events sometimes occur within genes, which can dysregulate endogenous gene expression.

To minimize integration events, integration-deficient LV vectors (IDLV) have been developed by mutating the HIV-1 Integrase component of LV vectors, to ensure that the majority of proviral DNA remains as extrachromosomal episomes. However, some chromosomal integration still occurs with IDLV technology, with 0.1-1% of proviruses integrating into the genome. This is potentially undesirable when persistence of the delivered gene has the potential to cause lasting harm to recipient cells, or when transgene expression needs to be controlled in a temporal manner.

Messenger RNA delivery offers a means to transiently express exogenous genes in a target cell, as the delivered mRNA remains extranuclear. Non-viral vectors have been developed for in vivo mRNA delivery, but delivery and tissue-specific targeting require further optimization. A variety of retroviral vectors have been engineered for transient delivery of their single stranded RNA (ssRNA) genomes for direct mRNA translation by mutating their reverse-transcriptase coding sequence. Additionally, viral vectors based on Sendai virus have been developed for in vivo delivery, but toxicity and limited efficacy preclude their clinical translation.

HIV-1-based LV vectors offer a potential means to deliver mRNA to a wide range of cell types in vivo and in vitro, as they package their genomes in the form of single-stranded RNA (ssRNA). Upon entering a cell, these ssRNA genomes are reverse-transcribed to give a double-stranded DNA product, which would then normally enter the nucleus. But it has been shown that HIV-1 reverse-transcriptase can be mutated to allow immediate translation of LV genomes upon cell entry. However, in previous attempts, this design has been ineffective in mediating gene therapy in ex vivo cultured haematopoietic stem cells (HSCs). Groups have further developed LV vectors for mRNA delivery engineering the bacteriophage MS2-Coat protein into LV capsids, and additionally engineered the MS2 RNA stem loop into LV genomic RNA. This MS2-mediated RNA delivery vector showed strong potential for gene delivery ex vivo and in vivo, although the functionality and production scalability of these chimeric LV-MS2 particles has not been investigated in detail.

The use of lentiviral vectors for mRNA delivery has potential advantages for applications requiring transient gene expression in a specific cell type. This includes in vivo and ex vivo gene editing applications, where it would be desirable to exploit vesicular stomatitis virus glycoprotein (VSVg) based transduction of target cell lines without long-term expression of gene editing nucleases. Additionally, the possibility to target antigen presenting cells with lentiviral vectors has been explored for vaccine development, although residual persistence of IDLV vector genomes could present a regulatory concern. Use of a transient mRNA delivery system in a lentiviral context could offer a significant development in these areas of research.

### SUMMARY OF THE INVENTION

The inventors of the present invention have developed a novel HIV-1-based LV vector that has been engineered for direct expression of its ssRNA payload upon target cell entry. The inventors have restructured and iteratively optimized the LV genome for this purpose, by relocating all HIV-1 material to the 3′ untranslated region, meaning that ribosomal entry occurs at the 7-methylguanylate (m7G) 5′ cap of the vector RNA. The inventors have demonstrated that this 5′ Cap-Dependent LV (CDLV) vector significantly improves the efficiency of LV ssRNA translation, compared to previously developed technologies that have depended on an internal ribosomal entry site (IRES) for translation. Furthermore, the inventors have shown that CDLV technology can deliver transient gene expression to mouse liver in vivo, matching the expression of DNA-based IDLV genomes over a 24-hour period. This introduces CDLV as a novel platform technology for potential use in transient treatment and manipulation of target cells.

Accordingly, in a first aspect of the invention, there is provided a retroviral RNA vector comprising a 5′ cap, a transgene, a 3′ long terminal repeat (LTR) and an RNA packaging sequence. Translation of the transgene is initiated at the 5′ end of the transgene in a cap-dependent manner. The 3′ LTR and the RNA packaging sequence are located 3′ of the transgene. The vector is not reverse transcribed into DNA in a target cell.

The retroviral vector provides the advantage that the RNA vector genome can be translated in a target cell without reverse transcription and integration of DNA into the target cell genome. This prevents aberrant integration events. Further, the retroviral vector is suitable for transient gene expression within a target cell.

Retroviral Vector

The retroviral vector can be based on any suitable retrovirus which is able to deliver genetic information to eukaryotic cells. Such vectors have been used extensively in gene therapy treatments and other gene delivery applications and are well known to those skilled in the art. For example, viruses which can be used in the preparation of the retroviral vector include lentiviruses, gamma-retroviruses (including murine leukaemia virus), alpha-retroviruses (including avian leukosis virus and Rous sarcoma virus), delta-retroviruses (including bovine leukaemia virus) and spumaretroviruses. In some embodiments, the retroviral vector may be a lentiviral vector. Preferably, the retroviral vector is a lentiviral vector based on HIV, in particular HIV-1.

The lentivirus group can be split into “primate” and “non-primate”. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

Details on the genomic structure of some lentiviruses may be found in the art. By way of example, details on HIV and EIAV may be found from the NCBI Genbank database (i.e. Genome Accession Nos. AF033819 and AF033820 respectively). Details of HIV variants may also be found at http://hiv.lanl.gov. Details of EIAV variants may be found through http://www.ncbi.nlm.nih.gov.

Each retroviral genome comprises genes called gag, pol and env which code for virion proteins and enzymes. Encapsidation of the retroviral RNAs occurs by virtue of a psi sequence located at the 5′ end of the viral genome.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction leads ultimately to infection by fusion of the viral membrane with the cell membrane.

Retroviruses may also contain “additional” genes which code for proteins other than gag, pol and env. Examples of additional genes include in HIV, one or more of vif, vpr, vpx, vpu, tat, rev and nef. EIAV has (amongst others) the additional gene S2.

The lentiviral vector of the present disclosure is a recombinant lentiviral vector. As used herein, the term “recombinant lentiviral vector” (RLV) refers to a vector with sufficient genetic information to allow packaging of an RNA genome, in the presence of packaging components, into a viral particle capable of infecting and transducing a target cell. The RLV carries non-viral coding sequences which are to be delivered by the vector to the target cell. An RLV is incapable of independent replication to produce infectious retroviral particles within the final target cell. Usually the RLV lacks a functional gag-pol and/or env gene and/or other genes essential for replication.

Preferably the recombinant lentiviral vector (RLV) of the present disclosure has a minimal viral genome. As used herein, the term “minimal viral genome” means that the viral vector has been manipulated so as to remove the non-essential elements and to retain the essential elements in order to provide the required functionality to infect, transduce and deliver a nucleotide sequence of interest to a target host cell.

However, the plasmid vector used to produce the lentiviral genome within a host cell/packaging cell will also include transcriptional regulatory control sequences operably linked to the lentiviral genome to direct transcription of the genome in a host cell/packaging cell. These regulatory sequences may be the natural sequences associated with the transcribed retroviral sequence, i.e. the 5′ U3 region, or they may be a heterologous promoter such as another viral promoter, for example the CMV promoter. Some lentiviral genomes require additional sequences for efficient virus production. For example, in the case of HIV, rev and RRE sequence are preferably included.

In one embodiment, the lentiviral vector is derived from a non-primate lentivirus. The non-primate lentivirus may be any member of the family of lentiviridae which does not naturally infect a primate and may include a feline immunodeficiency virus (FIV), a bovine immunodeficiency virus (B1V), a caprine arthritis encephalitis virus (CAEV), a Maedi visna virus (MW) or an equine infectious anaemia virus (EIAV). Non-primate lentiviral-based vectors do not introduce HIV proteins into individuals.

Cap

The 5′ cap is well known to those skilled in the art as a specially altered nucleotide on the end of a primary transcript. The 5′ cap is vital in the creation of stable and mature messenger RNA (mRNA) able to undergo translation during protein synthesis. The 5′ cap interacts with initiation factors which then bind other proteins to form the pre-initiation complex. This pre-initiation complex scans the RNA and once it reaches the start codon of the transgene, the ribosome assembles and translation is initiated. This process is known as cap-dependent initiation. The 5′ cap is also important in the regulation of nuclear export, prevention of degradation by exonucleases and promotion of 5′ proximal intron excision. Appropriate 5′ caps which can be used to initiate translation of RNA are well known to those skilled in the art and include m7G, GpppG, m7GpppG, m7GpppGm, m7GpppNm, m2,2,7GpppG, m7,3′-OGpppG, cap-1, cap-2, m6Am, NAD+, reduced NAD+ (NADH) and 3′-dephospho-coenzyme A (dpCoA). In some embodiments, the 5′ cap is selected from m7G, GpppG, m7GpppG, m7GpppGm, m7GpppNm, m2,2,7GpppG, m7,3′-OGpppG, cap-1, cap-2 and m6Am. In some embodiments, the 5′ cap is a 5′ m7G cap. The 5′ m7G cap structure consists of a 7-methylguanosine triphosphate linked to the 5′ end of the mRNA via a 5′ to 5′ triphosphate linkage.

Preferably, the vector does not comprise an internal ribosome entry site (IRES) sequence. In conventional vectors, IRES sequences are commonly located in an internal region of the transgene cassette and facilitate translation of the RNA in a cap-independent manner. In certain vectors of the prior art, IRES sequences are located immediately upstream of a transgene to facilitate translation of the RNA in a cap-independent manner. In contrast, the vectors of the present application have been engineered so that an IRES sequence is not required for translation of the transgene. This has the advantage that translation of packaged genomic mRNA is enhanced immediately upon cell entry.

Transgene

The vector comprises a transgene for delivery into a target cell. This transgene may be any transgene which someone might want to transiently express in a target cell. This transgene may be any transgene which someone does not want to integrate within the target cell genome. The transgene is located close to the 5′ cap such that translation of the transgene is initiated at the 5′ end of the transgene in a cap-dependent manner. Preferably, the transgene is located adjacent to the 5′ cap. “Adjacent” is defined as being next to or adjoining something else. In this context, the transgene is located next to the cap in the vector, such that translation is initiated at the 5′ end of the transgene in a cap-dependent manner. Cap-dependent initiation of translation in eukaryotes has been well documented in the art.

The transgene may encode for a peptide or protein. In the present invention, the transgene is not under the control of a promoter (e.g. a PGK or GAPDH promoter).

Transient expression of the transgene may facilitate editing of the genome of a target cell. For example, the transgene may encode for an engineered nuclease, such as a zinc finger nuclease (ZFN), a Transcription Activator-like Effector Nuclease (TALEN) or a Clustered Regularly Interspaced Short Palindromic Repeats (CRISPR)-associated (Cas) nuclease. In some embodiments, the transgene encodes Cas9 nuclease for in vivo and ex vivo gene editing applications. Transient expression of these nucleases is advantageous to avoid continuous or long-term expression in target cells that may be toxic. The packaging capacity of retroviral vectors means that the technology has the potential to express large therapeutic transcripts transiently and that Cas9 expression transcripts may be easily modified to include any of the recently developed modules (e.g. base editors, transcriptional activators and/or repressors), without compromising packaging ability.

Transient expression of the transgene may contribute to inducing a specific immune response. For example, transient expression of an antigen in an antigen-presenting cell (APC) may lead to processing and presentation of the antigen on the surface of the APC. Presentation of antigen to immune cells (such as T cells and B cells) may lead to an antigen-specific immune response. This application may be directed to anti-pathogen immune responses (e.g. vaccines), and/or anti-cancer immune responses. A lack of pre-existing immunity to retroviruses means that retroviral vectors may allow for repeated administrations (e.g. prime-boost regimes, re-dosing, etc.). A current issue with conventional vectors utilising reverse transcription for APC transduction is the expression of SAMHD1 (SAM and HD domain-containing deoxynucleoside triphosphate triphosphohydrolase 1) in these immune cells which lowers intracellular dNTP pools and reduces transduction efficiency by restricting the activity of reverse-transcriptase. As the vectors of the present application do not depend on reverse transcriptase activity, this issue is circumvented.

Transient expression of the transgene may aid normal growth of the cell or maintain the health of a subject. In some embodiments, the transgene encodes for a peptide or protein which is absent or underexpressed in a subject. Alternatively, the transgene may encode for a peptide or protein which helps to prevent or ameliorate a medical condition. The peptide or protein may be one which is useful in treating diseases such as cancer, atherosclerosis, sickle-cell anaemia, infection, metabolic disorders, neurological illness and the thalassemias. Examples of such peptides and proteins are haemoglobin, hematopoietic growth factors such as granulocyte-macrophage colony stimulating factor (GM-CSF), macrophage colony stimulating factor (M-CSF), granulocyte colony stimulating factor (G-CSF), erythropoietin (EPO), common gamma chain, Wiskott Aldrich Syndrome protein (WASp), GP91phox, and ABCD1. Another example is tumour necrosis factor (TNF), which is a molecule that can be used to treat cancer, and in particular, tumours. The tumour suppressor p53 and retinoblastoma (RB) are also contemplated. Various cytokines such as mast cell growth factor (MGF) and interleukins 1-11 are also proteins which are contemplated by the present invention. A multidrug resistance gene (mdR) encoding p-glycoprotein is also contemplated as the transgene. The peptide or protein may also be a selectable marker for antibiotic resistance in eukaryotes. Other types of selectable markers such as adenine phosphoribosyl transferase (APRT) in APRT-deficient cells, a fluorescent protein or the firefly luciferase gene are also included. The peptide or protein can be a protein that will provide the host with an additional or altered enzymatic activity, such as the herpes simplex virus thymidine kinase protein for ‘suicide therapy’ of reactive transplants, or a toxin, such as the diphtheria toxin protein for treatment of cancer. The transgenes encoding these proteins can be provided by any of a variety of methods, such as routine cloning procedures (Sambrook et al. (1989), Molecular Cloning: A Laboratory Manual, Cold Spring Harbor, NY), excision from a vector containing the gene of interest, or chemical or enzymatic synthesis based on published sequence information. In many instances the DNA encoding the protein of interest is commercially available. In another embodiment, the transgene encodes a protein which enables experimental manipulation of the cell, for example a toxin or a fluorescent or drug-selectable marker.

In some embodiments, the vector is provided at regular intervals such that continual or near-continual expression of the transgene is provided. In these embodiments, the transgene may be any transgene where continual expression of the transgene in the target cell is desired.

The vector may further comprise an intron. The presence of an intron may help with RNA export. In some embodiments, the intron is located on the 5′ side of the transgene, in between the 5′ cap and the transgene. In this instance, translation is initiated at the 5′ end of the transgene coding sequence. The pre-initiation complex forms at the 5′ cap and scans through the intron before reaching the start codon of the transgene, where the ribosome assembles to initiate translation. In this way, the presence of the intron does not prevent cap-dependent initiation and translation of the transgene.

In some embodiments, the intron is 3′ of the transgene. In various embodiments, the intron is 3′ of the transgene and 5′ of the 3′ LTR. In particular embodiments, the intron is immediately downstream (3′) of a polyadenylation signal. The intron may be any length that allows packaging of the vector genome. Preferably, the intron is a short sequence.

In some embodiments, the first start codon (i.e. the start codon positioned first in a 5′ to 3′ direction of the vector) encountered by the pre-initiation complex is the start codon of the transgene.

In some embodiments, the vector comprises an IRES sequence. In some embodiments, the IRES sequence is located on the 5′ side of the transgene, in between the 5′ cap and the transgene. In this instance, translation is initiated at the 5′ end of the transgene coding sequence in a cap-dependent manner. The pre-initiation complex forms at the 5′ cap and scans along the RNA to reach the start codon of the transgene, where translation is initiated. In this way, the presence of the IRES does not prevent cap-dependent initiation and translation of the transgene.

The transgene may further comprise a Kozak sequence, preferably a strong Kozak sequence. The Kozak sequence is a nucleic acid motif that functions as the protein translation initiation site in most eukaryotic mRNA transcripts. Variation within the Kozak sequence alters the “strength” thereof. Kozak sequence strength refers to the favorability of initiation, affecting how much protein is synthesised from a given mRNA. Optimal, strong, moderate and weak Kozak sequences have been well documented and a skilled person would be able to determine the relative strength of the motif as described in the art (Hernandez et al., Conservation and Variability of the AUG Initiation Codon Context in Eukaryotes, Trends in Biochemical Sciences, Vol. 44, Issue 12, 2019, Pages 1009-1021).

3′ LTR

The vector comprises a 3′ long terminal repeat (LTR) that is located 3′ of the transgene. Retroviral LTRs are generally segmented into U3, R, and U5 regions. However, in certain LTRs, parts of these regions may be deleted. The term “long terminal repeat” or “LTR” is intended to cover all such variations in LTRs. LTRs can comprise a number of signals required for gene expression such as a transcriptional enhancer, a promoter, a transcription initiation signal and/or a polyadenylation signal.

The 3′ LTR may be a self-inactivating (SIN) LTR. In the vector, for enhanced safety the 3′ LTR is preferably a self-inactivating LTR in which nucleotides in the U3 region have been deleted. This can include the TATA box and binding sites for transcription factors. SIN LTRs are well known to those skilled in the art (e.g. see Retroviruses. Edited by Coffin J M, Hughes S H, and Varmus H E. Cold Spring Harbor (NY): Cold Spring Harbor Laboratory Press; 1997).

RNA Packaging Sequence

The vector comprises an RNA packaging sequence that is located 3′ of the transgene. The RNA packaging sequence is necessary for the essential process of packaging the retroviral RNA genome into the viral particle as it is assembled by the producer cell. The RNA packaging sequence is able to bind to viral proteins within the nascent viral particle.

In some embodiments, the RNA packaging sequence comprises the RNA packaging signal (Ψ). In HIV-1, a portion of the gag gene has been found to be involved in RNA packaging. In some embodiments, the RNA packaging sequence comprises a portion of the gag gene (i.e. a truncated gag gene). In various embodiments, the RNA packaging sequence comprises a portion of the gag gene. The RNA packaging sequence may also comprise the Rev Response Element (RRE). RNA packaging sequences and the components that make this up are well known to those skilled in the art (e.g. see Retroviruses. Edited by Coffin J M, Hughes S H, and Varmus H E. Cold Spring Harbor (NY): Cold Spring Harbor Laboratory Press; 1997).

The RNA packaging sequence may be located 3′ of the 3′ LTR. The RNA packaging sequence may be located at the 3′ end of the vector.

Preferably, the RNA packaging sequence comprises the Ψ, a portion of the gag gene and the RRE. More preferably, the RNA packaging sequence comprises the Ψ, a portion of the gag gene and the RRE, and is located 3′ of the 3′ LTR. The Ψ may partially overlap the translation start codon of the gag gene.

Other Features

In some embodiments, the vector does not comprise a 5′ LTR. In some embodiments, the vector does not comprise a central polypurine tract (cPPT).

In various embodiments, the vector does not comprise a 5′ LTR located upstream (i.e. on the 5′ side) of the transgene. In particular embodiments, the vector does not comprise a packaging sequence located upstream of the transgene. In particular embodiments, the vector does not comprise a Ψ, a portion of the gag gene and/or an RRE located upstream of the transgene. In some embodiments, the vector does not comprise a cPPT located upstream of the transgene.

In some embodiments, the vector does not comprise a primer binding site (PBS). In conventional vectors, the PBS is a site which binds to a tRNA primer which is responsible for initiating minus strand synthesis during the reverse transcription process. However, in the present invention, reverse transcription of the vector genome does not occur and a PBS is not required. In some embodiments, the vector comprises a mutated reverse transcriptase enzyme.

The vector may comprise further elements which help to improve the stability and translation of the vector. These elements are generally located downstream of the transgene, either before or after the packaging sequence.

For example, the vector may further comprise a post-transcriptional regulatory element (PRE) such as a woodchuck hepatitis virus post-transcriptional regulatory element (WPRE). In some embodiments, the PRE, optionally the WPRE, is located immediately downstream (3′) of the transgene.

The vector may further comprise a nuclear export signal, such as the constitutive transport element (CTE) and/or the RNA transport element (RTE). CTEs belong to type D retroviruses and function in nuclear export of partially spliced mRNAs. RTEs enhance cellular mRNA export in rodent intracisternal A-particle (IAP) retroelements. Unlike the HIV RRE, CTEs and RTEs utilise host factors for RNA export. Previous research has shown that CTEs and RTEs can be used in place of RRE in lentiviral vectors to avoid use of Rev, whilst a combination of CTEs and RTEs has been shown to enhance lentiviral vector RNA turnover.

The vector may further comprise one or more polyadenylation (polyA) signals such as the late polyA, the simian virus 40 (SV40) early polyA, the bovine growth hormone polyA (bGHpA), the human growth hormone polyA (hGHpA) and the rabbit beta-globin polyA (rBGpA) signals. Preferably, the polyA signal is located downstream (3′) of the transgene. More preferably, the polyA signal is located downstream (3′) of the PRE, optionally the WPRE.

In various embodiments, the vector comprises, in 5′ to 3′ direction, the following components:
(i) 5′-transgene-3′ LTR-RNA packaging sequence-3′
(ii) 5′-transgene-PRE-3′ LTR-RNA packaging sequence-3′
(iii) 5′-transgene-PRE-3′ LTR-ψ-ΔGag-RRE-3′
(iv) 5′-transgene-PRE-polyA-3′ LTR-RNA packaging sequence-3′
(v) 5′-transgene-PRE-polyA-intron-3′ LTR-RNA packaging sequence-3′
(vi) 5′-intron-transgene-3′ LTR-RNA packaging sequence-3′
(vii) 5′-intron-transgene-3′ LTR-ψ-ΔGag-RRE-3′

All of the above vectors are also envisaged as including a 3′ poly(A) tail.

Also envisaged as part of the disclosure are the nucleotide sequences encoding the vector genomes described herein. Also envisaged as part of the disclosure are the nucleotide sequences encoding the vector genomes described herein, the nucleotide sequences further comprising a promoter operably linked to the vector genome, and a poly(A) signal.

Also envisaged as part of the disclosure are the DNA plasmids encoding the vector genomes described herein. Also envisaged as part of the disclosure are the DNA plasmids encoding the vector genomes described herein, the DNA plasmids further comprising a promoter operably linked to the vector genome, and a poly(A) signal.

Accordingly, in a second aspect of the invention, there is provided a nucleotide sequence encoding a vector genome, the nucleotide sequence comprising a promoter and a vector genome. The promoter is operably linked to the vector genome. The vector genome comprises a transgene, a 3′ LTR and an RNA packaging sequence. The transgene is located at the 5′ end of the vector genome such that, following transcription and capping of the vector genome, translation is initiated at the 5′ end of the transgene in a cap-dependent manner. The 3′ LTR and the RNA packaging sequence are located 3′ of the transgene. The vector genome is not reverse transcribed into DNA in a target cell.

The promoter is operably linked to the vector genome such that the promoter drives transcription of the vector genome. The promoter may be any suitable promoter, including a human cytomegalovirus (CMV) immediate early promoter, a Rous Sarcoma Virus (RSV) promoter, a spleen focus forming virus (SFFV) promoter or an HIV-1 U3 promoter. The promoter is preferably positioned at the 5′ end of the nucleotide sequence.

The nucleotide sequence may further comprise a poly(A) signal, such as the simian virus (SV40) early polyA signal, the late polyA signal, and the bovine growth hormone polyA (bGHpA) signal. Preferably, the vector further comprises an SV40 polyA signal. Preferably, this is located at the 3′ end of the nucleotide sequence after the vector genome.

In various embodiments, the nucleotide sequence comprises, in 5′ to 3′ direction, the following components:
(i) 5′-promoter-transgene-3′ LTR-RNA packaging sequence-poly(A) signal-3
(ii) 5′-promoter-transgene-PRE-3′ LTR-RNA packaging sequence-poly(A) signal-3′
(iii) 5′-promoter-transgene-PRE-3′ LTR-ψ-ΔGag-RRE-poly(A) signal-3′
(iv) 5′-promoter-transgene-PRE-polyA-3′ LTR-RNA packaging sequence-poly(A) signal-3′
(v) 5′-promoter-transgene-PRE-polyA-intron-3′ LTR-RNA packaging sequence-poly(A) signal-3′
(vi) 5′-promoter-intron-transgene-3′ LTR-RNA packaging sequence-poly(A) signal-3′
(vii) 5′-promoter-intron-transgene-3′ LTR-ψ-ΔGag-RRE-poly(A) signal-3

There is also provided a nucleotide sequence according to SEQ ID NO: 1. There is also provided a nucleotide sequence according to SEQ ID NO: 2. There is also provided a nucleotide sequence according to SEQ ID NO: 3. There is also provided a nucleotide sequence according to SEQ ID NO: 4. There is also provided a nucleotide sequence according to SEQ ID NO: 5. There is also provided a nucleotide sequence according to SEQ ID NO: 6. There is also provided a nucleotide sequence with at least 99%, 98%, 97%, 96%, 95%, 90%, 85%, 80% identity to SEQ ID NO: 1. There is also provided a nucleotide sequence with at least 99%, 98%, 97%, 96%, 95%, 90%, 85%, 80% identity to SEQ ID NO: 2. There is also provided a nucleotide sequence with at least 99%, 98%, 97%, 96%, 95%, 90%, 85%, 80% identity to SEQ ID NO: 3. There is also provided a nucleotide sequence with at least 99%, 98%, 97%, 96%, 95%, 90%, 85%, 80% identity to SEQ ID NO: 4. There is also provided a nucleotide sequence with at least 99%, 98%, 97%, 96%, 95%, 90%, 85%, 80% identity to SEQ ID NO: 5. There is also provided a nucleotide sequence with at least 99%, 98%, 97%, 96%, 95%, 90%, 85%, 80% identity to SEQ ID NO: 6.

All of the embodiments that apply to the first aspect equally apply to the second aspect and vice versa.

In a third aspect, there is provided a host cell containing the vector described above. The host cell may be any suitable eukaryotic cell into which the vector may be introduced. The host cell may be a mammalian cell or a plant cell. The host cell may be a human cell. The host cell may be in vivo, in vitro or ex vivo.

Plasmids encoding the retroviral vectors of the present invention are transfected into suitable host cells (or packaging cells) by standard methods known to one of ordinary skill in the art. Suitable packaging cells are defined herein as cells that contain helper virus sufficient to allow the packaging of RNA transcribed from the retroviral vector and the release of vector virus particles, or virions. Generally additional plasmids encoding trans-acting viral sequences but lacking the cis-acting sequences required for packaging are co-transfected. These supply the required structural and enzymatic proteins to package and produce the expressed viral backbone RNA. Such packaging cells are known and available to one of ordinary skill in the art, and include, for example, HEK293T cells.

Recombinant retrovirus produced from the transfected cells is harvested by standard methods. The harvested retrovirus, in the form of virions, is used to transduce a permissive target cell by standard techniques. A target cell is defined herein as any cell that is permissive to infection by the virus produced by the retroviral vector of the present invention. The target cell can be in vivo or ex vivo. Representative target cells include, for example, antigen presenting cells, bone marrow stem cells, hepatocytes, muscle cells, tumour cells, neurons, retina and airway epithelial cells.

In a fourth aspect, there is provided a virion containing the vector described above.

In a fifth aspect, there is provided a pharmaceutical composition comprising the vector or virion described above. The pharmaceutical composition may further comprise one or more pharmaceutically acceptable excipients.

In a sixth aspect, there is provided a vector or virion for use in therapy, in particular, gene therapy.

In a seventh aspect, there is provided a vector or virion for use in delivering a transgene to a subject in gene therapy.

In an eighth aspect, there is provided a method of delivering a transgene to a target cell, the method comprising administering an effective amount of a vector or virion to the target cell. The method does not comprise a step of reverse transcription of the vector genome into DNA. This method can be used to transiently express a transgene within the target cell, without reverse transcription of the vector genome or integration of the transgene into the target cell genome. For example, the method can be used to transiently express an engineered nuclease, such as Cas9, in a target cell such that Cas9 protein is produced in the target cell without the vector entering the nucleus.

In a ninth aspect, there is provided a method of delivering a transgene to a target cell in a subject, the method comprising administering an effective amount of a vector or virion to the subject. The method does not comprise a step of reverse transcription of the vector genome into DNA. The subject may be human or animal. Where the subject is human, the transgene can be delivered to the subject as part of gene therapy so that the transgene is expressed in the subject.

In a tenth aspect, there is provided a cell produced by the above method. The cell may be any cell that is permissive to infection by the virus produced by the retroviral vector of the present invention. The cell can be in vivo or ex vivo. Representative cells include, for example, antigen presenting cells, bone marrow stem cells, hepatocytes, muscle cells, tumour cells, neurons, retina and airway epithelial cells.

All of the embodiments that apply to the first aspect and second aspect equally apply to the third, fourth, fifth, sixth, seventh, eighth, ninth and tenth aspects.

The invention will now be described in detail, by way of example only, with reference to the following figures.

### BRIEF DESCRIPTION OF THE DRAWINGS

FIG. 1 is a schematic showing lifecycle of LV vectors designed for direct mRNA expression. a) In the left panel, a conventional LV transduces the cell and reverse-transcribes its ssRNA into dsDNA, which then enters the nucleus to either integrate into the host genome or exist as a circular ‘episome’. The nuclear DNA can then use host machinery to drive transgene mRNA production and express the transgene protein. On the right panel, RT-deficient LV vectors contain a mutated reverse-transcriptase (RT) enzyme, which means that packaged ssRNA is free to interact with cell ribosomes to express contents as mRNA. b) Vector schematics show the structure of the DNA template used to generate vector genome RNA. White boxes indicate elements that are present in DNA alone, whilst grey boxes indicate the elements that are transcribed into the encapsidated RNA genomes. ‘IRES-mediated’ mRNA expression has previously been reported, where the vector RNA genome contains an internal IRES which allows translation of the downstream coding sequence (in this case EGFP). With the inventors' novel ‘Cap-mediated’ mRNA expression system, the transgene coding sequence is positioned at the extreme 5′ terminus of vector ssRNA, meaning that translation initiation occurs from the 5′ m7G cap. Additionally, the vector lacks a HIV-1 primer binding site, further reducing any probability of the mutated reverse-transcriptase binding its canonical target.

FIG. 2 identifies the optimal genome structure for CDLV translation. a) Six versions of CDLV (CDLV1-6) were developed, with designs aimed to enhance RNA processing for optimal packaging and translation upon cell entry. The schematics represent the structure of lentiviral vector RNA genomes that would be packaged into vector particles. LTR, HIV-1 long terminal repeat; Δ Gag, truncated and inactive HIV-1 gag gene, including Ψ packaging sequence; RRE, HIV-1 rev response element; cPPT, HIV-1 central polypurine tract; IRES, internal ribosome entry site (IRES) of the encephalomyocarditis virus; EGFP, enhanced green fluorescent protein; WPRE, woodchuck-hepatitis virus post-transcriptional regulatory element; Intron, chimera between introns from human β-globin and immunoglobulin heavy chain genes; bGHpA, bovine growth hormone polyadenylation sequence; U5, U5 domain of the HIV-1 LTR. b) Comparison of functional titres gained from each of the ssRNA genomes. Titres were determined by the percentage of EGFP+ cells in transduced cells, indicating transduction units per milliliter (TU/ml). Statistical comparison of the titres was made by Kruskal Wallis test with Dunn's posthoc analysis (*P<0.001). Averages were calculated from 4 experimental replicates in each case.

FIG. 3 shows the comparison of CDLV transduction kinetics versus IRES-based RT-deficient vectors. a) IRES-EGFP and CDLV-EGFP vectors were produced using the depicted genomes. b) HEK 293T cells were transduced with the vectors. Longitudinal analysis of EGFP expression shows CDLV vector expression profile compared versus the IRES-based system over the course of the experiment (P=0.0196 by t-test). c) Flow cytometry plots show EGFP mean fluorescence intensity (MFI) per transduced cell, comparing CDLV-EGFP expression versus IRES-EGFP. d) Vectors dosed by p24 capsid protein mass allow crude estimation of the number of vector capsids required to achieve optimal gene expression. This analysis demonstrates that 1 ng p24 per cell is necessary to achieve optimal expression intensity with CDLV (approximately 1×10⁷ LV particles).

FIG. 4 shows the comparison of CDLV transduction kinetics to conventional IDLV and IPLV vectors. a) Integration deficient (IDLV-SFFV-EGFP) and integration-proficient (IPLV-SFFV-EGFP) lentiviral vectors containing an SFFV-EGFP expression cassette were compared to a CDLV-EGFP vector. b) HEK 293T cells transduced and the percentage of EGFP+ cells was tracked for 2 weeks, to monitor expression longevity. c) The intensity of EGFP expression gained from each vector is shown for the first 72 hours after transduction. d) The percentage of EGFP-positive cells is compared for CDLV-EGFP at 24-hours post-transduction and IDLV-SFFV-EGFP at 72-hours post-transduction (the relative timing of peak expression for each vector platform) (P=0.05 by t-test). e) The same data set shown in d) was analyzed for EGFP fluorescence (MFI), to compare the strength of EGFP expression per transduced cell (P=0.005 by t-test).

FIG. 5 tracks the in vivo expression kinetics of CDLV compared to DNA-based LV vectors. a) IDLV or IPLV vectors were produced containing a bicistronic luciferase-EGFP expression cassette, driven by the SFFV promoter (IPLV/IDLV-SFFV-Luc-EGFP). A CDLV vector was additionally produced containing the same ‘Luc-EGFP’ coding sequence (CDLV-Luc-EGFP). b) In vivo bioluminescence was imaged at various time-points for 10 days (240 hours) after vector injection, to track the onset and persistence of transgene expression. Data are expressed as photons per second per cm 2 per steradian (n=4 mice per group). c) The associated heat maps for each mouse indicate bioluminescence signal intensity over the initial 96-hours of the experiment.

### DETAILED DESCRIPTION OF THE INVENTION

### Example 1

Results

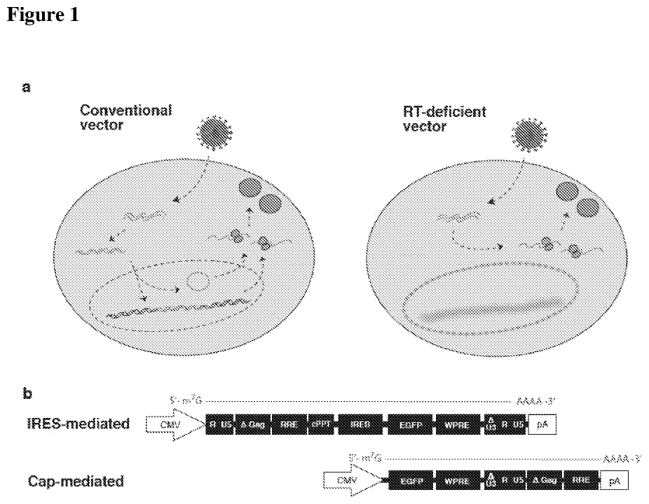

The aim of this investigation was to develop an LV-based vector that can efficiently deliver its genome as mRNA to target cells in vivo and ex vivo. It has previously been reported that the HIV-1 Reverse-Transcriptase component of LV vectors can be mutated to remove its ability to convert RNA into DNA. This reverse transcription deficient (RT-deficient) LV vector platform then achieves transgene expression in target cells without forming a DNA intermediate (FIG. 1a).

Engineering LV Vector Genomes to Maximize Translation of Packaged ssRNA

In previous studies, RT-deficient vectors have achieved gene expression in a subset of target cells, but the expression level was insufficient to mediate targeted disruption of the human CCR5 gene in HSCs. The inventors hypothesized that this lack of efficacy was due to reliance on an IRES element to initiate translation, due to the presence of 1.5 kb wild-type HIV-1 DNA in the 5′ region of the vector RNA (FIG. 1b).

In order to eliminate this problem, the inventors restructured the vector genome by moving the 1.5 kb HIV-1 DNA downstream of the therapeutic transgene (FIG. 1b). This configuration was designed to leave a Kozak consensus sequence at the extreme 5′ terminus of the vector RNA, which would be 5′ m⁷G cap capped during vector production. Additionally, the HIV-1 primer-binding site (PBS) was completely removed to eliminate canonical reverse-transcriptase priming. The inventors' expectation was that the 5′ m⁷G cap-mediated LV vector would produce a greater level of gene expression than the IRES-mediated configuration.

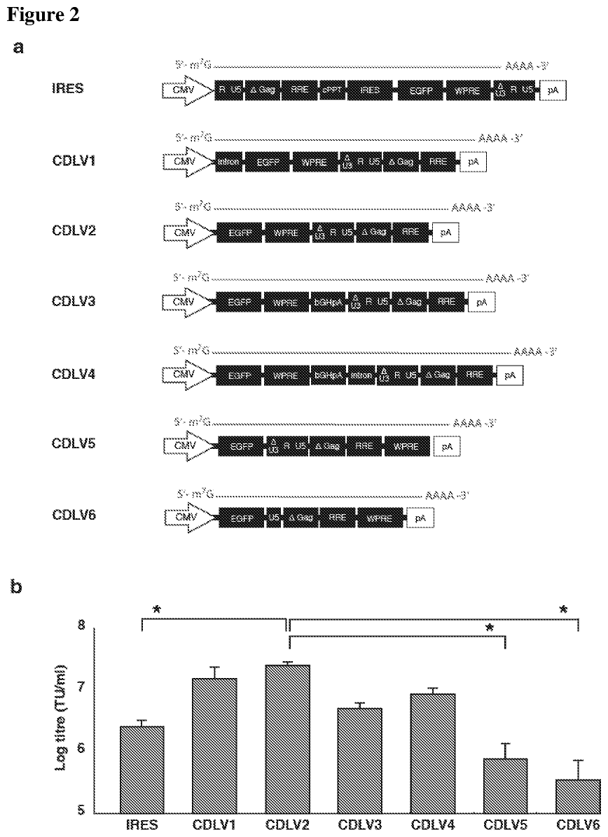

The inventors engineered six iterations of the cap-mediated ‘CDLV’ vector, with the aim of identifying a configuration that could exceed IRES-based LV expression (FIG. 2a). In each case, the depicted vector RNA is derived from a plasmid using the Cytomegalovirus (CMV) promoter to transcribe vector genome RNA. Each variant contained variable combinations of non-coding domains of the transcribed region. Version 1 (CDLV1) was designed to include a chimeric intron (fusion of introns from human (3-globin and immunoglobulin heavy chain genes), based in the expectation that the presence of splice sites at the 5′ end would enhance nuclear export of the RNA. CDLV2 was identical to version CDLV1, but for omission of the chimeric intron. CDLV3 included a polyA motif upstream of the 3′LTR to enhance LV ssRNA expression, whilst CDLV4 additionally contained an intron downstream of the polyA, to minimise premature termination during vector production. Finally, CDLV5 and CDLV6 aimed to promote read-through of the 3′LTR by relocating WPRE to the extreme 3′ terminus and deleting portions of the U3-R domains that contain HIV-1 polyA motifs.

In each case, vectors were designed to deliver an enhanced green fluorescent protein (EGFP) RNA payload. Vector titres were determined by transduction of HEK 293T cells and quantifying the number of EGFP-positive cells by flow cytometry. This comparison revealed that CDLV version 2 yielded titres one order of magnitude greater than the IRES-based vector (P<0.001) (FIG. 2b). This structure was therefore taken forwards for further characterization of the technology.

Cap-Dependent Translation Provides Greater Gene Expression than IRES-Dependent Translation

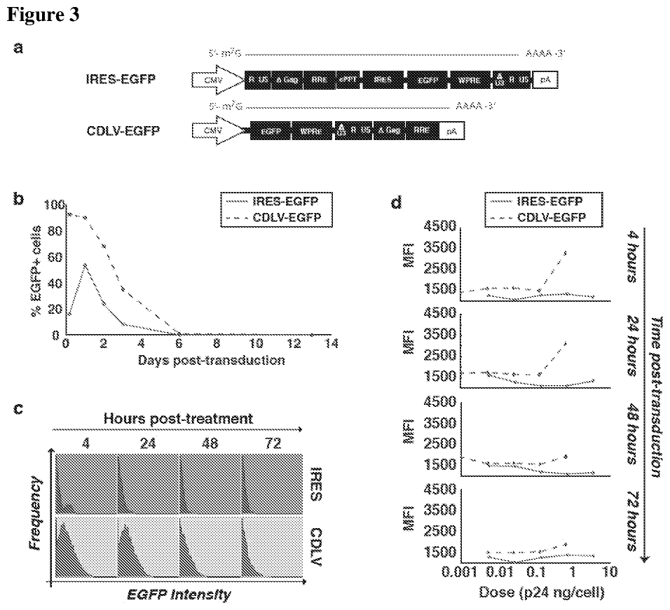

The transduction kinetics of CDLV version 2 were compared in greater detail versus the RES-mediated system. HEK 293T cells were transduced with an equal dose of either CDLV-EGFP or IRES-EGFP (FIG. 3a). Longitudinal analysis of EGFP expression showed that the CDLV vector produced a greater number of EGFP-positive cells than IRES-based LV throughout the course of the experiment (P=0.0196), with both vectors achieving peak expression by 24 hours and dropping to minimal levels by day 6 (FIG. 3b). EGFP fluorescence intensity was also quantified at each timepoint, with CDLV expression exceeding that of IRES-based vectors up to 72-hours post-transduction (P<(FIG. 3c).

Additionally, in order to clarify that these expression values were being derived from enhanced expression, rather than excess physical particle mass, dose response profiles were also compared in investigations in which vectors were dosed by capsid mass. These investigations showed that the CDLV vector gave the greatest level of EGFP expression at 4 hours and 24 hours post-transduction, when administered at a dose of 0.64 ng p24/cell. The IRES-based vector was unable to match this level, even when administered to cells at higher doses of 0.7 ng p24/cell and 3.5 ng p24/cell (FIG. 3d). The intensity of EGFP expression from IRES vectors was not enhanced at any of the tested doses, indicating that much higher doses would be needed to accumulate equivalent EGFP levels as those observed for CDLV.

CDLV Vectors Provide a Transient Burst of Gene Expression In Vitro

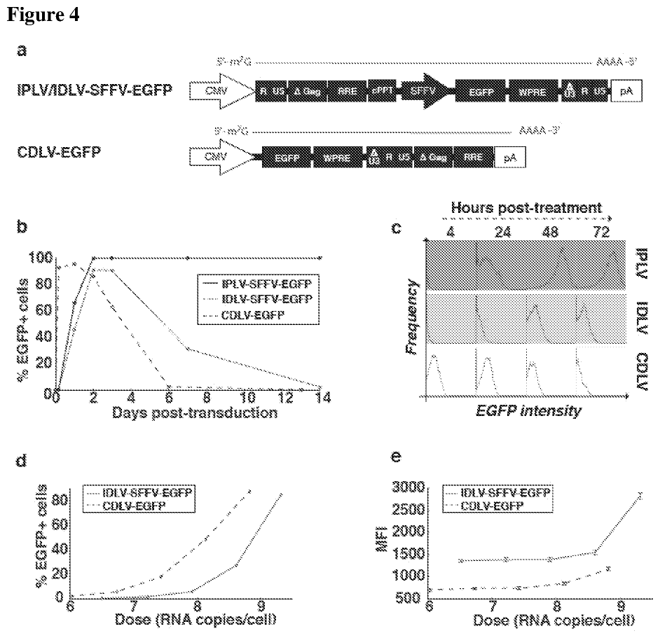

After demonstrating the potential advantages of CDLV as an mRNA delivery platform, the inventors set out to investigate how its longitudinal expression profile compared to DNA-based gene delivery systems. In this experiment, EGFP was again employed as a transgene, driven by the spleen focus-forming virus (SFFV) promoter in the DNA-based IPLV and IDLV vectors (FIG. 4a).

CDLV-EGFP was delivered to HEK 293T cells at a multiplicity of infection (MOI) of 41 EGFP-forming units per cell (EFU/cell), whilst IPLV-SFFV-EGFP and IDLV-SFFV-EGFP were delivered at doses of 10 EFU/cell. As expected, CDLV produced a transient expression profile, with peak expression occurring around 24 hours post-transduction, matching the longitudinal profile seen with previous retrovirus-based mRNA delivery platforms. whereas IPLV and IDLV vectors peaked at around 48 hours post-transduction, with the IDLV profile falling to 2.2% EGFP-positive cells by day 14 (FIG. 4b). Analysis of mean fluorescence intensity again showed that CDLV expression appeared sooner than IDLV and IPLV, with CDLV expression level remaining detectable throughout the initial 72 hours post-transduction (FIG. 4c). Quantification of vector DNA in the transduced cells did not reveal presence of proviruses in any of the CDLV-transduced cells (data not shown), confirming that the RT mutation has eliminated RT functionality.

Data presented in FIG. 4b and FIG. 4c appear to show that CDLV expression peaks at 24 hours post-transduction, whereas IDLV expression peaks at 72 hours. To compare their relative gene expression efficacy at these time-points, CDLV and IPLV vectors were titered by RNA genome copy number and administered to HEK 293T cells at known vector genome doses, with EGFP expression measured at their respective peak time-points. This investigation revealed that CDLV produced a similar number of EGFP-positive cells to IDLV, even when administered at a lower RNA copy number (P=0.05) (FIG. 4d). However, when examining the intensity of EGFP expression in the transduced cells, it was seen that IDLV treatment produced a greater level of expression per administered RNA genome (FIG. 4e) (P=0.005), likely due to the use of a strong SFFV promoter in the IDLV format, leading to mRNA abundance.

LV Vectors can Express ssRNA Payloads in Mouse Liver In Vivo

LV vectors are commonly pseudotyped with VSVg, a glycoprotein that confers broad tissue tropism by targeting the low-density lipoprotein receptor (LDLR) for cell entry (Finkelshtein, D et al. (2013), Proc. Natl. Acad. Sci. U.S.A 110: 7306-11). VSVg-pseudotyped LV vectors are particularly effective for in vivo liver transduction (Pan, D et al. (2002), Mol. Ther. 6: 19-29). The inventors investigated the effectiveness of their engineered CDLV vector for gene transfer to neonatal mouse liver in vivo, comparing its longitudinal expression profile to a conventional IDLV vector.

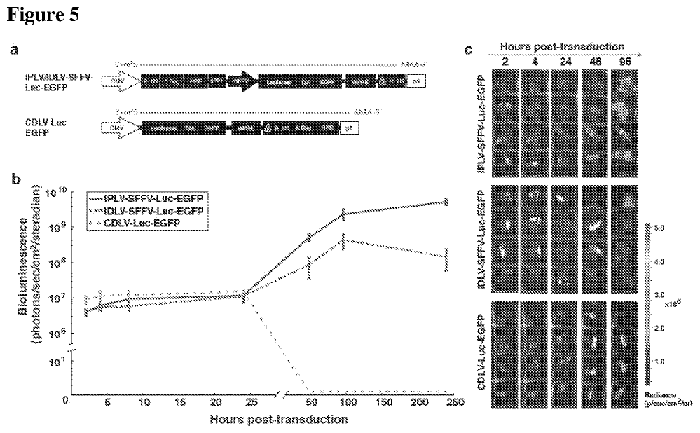

The inventors used a bicistronic transgene expressing luciferase and EGFP, separated by a 2A cleavage peptide derived from Thosea asigna virus (Szymczak, A L et al. (2004), Nat. Biotechnol. 22: 589-594). This ‘Luc-EGFP’ reporter was packaged into IPLV and IDLV vectors driven by the SFFV promoter (IPLV-SFFV-Luc-EGFP and IDLV-SFFV-Luc-EGFP, respectively). The Luc-EGFP transgene was additionally packaged into a CDLV vector (CDLV-Luc-EGFP) (FIG. 5a). Neonatal outbred mice received intravenous injections of each vector on the day of birth and bioluminescent imaging began 2 hours after vector administration and continued for days (FIGS. 5b and 5c). Vector doses were calculated based on ssRNA titres, with IPLV being delivered at 1×10¹³ viral genomes per milliliter (vg/ml), IDLV delivered at 5×10¹² vg/ml and CDLV delivered at 4×10¹¹ vg/ml. As expected, IPLV vector expression was detectable at early stages post-transduction and expression intensity continued to increase throughout the course of the investigation. IDLV vectors showed reduction of bioluminescent signal after 96 hours post-transduction, indicating reduced persistence in transduced liver. CDLV vector expression was detectable by 2 hours post-injection, with its expression appearing to peak at 24 hours before falling below the limit of detection thereafter.

Discussion

Lentiviral vectors are effective gene transfer agents, with an ability to transduce a variety of cell types in vitro and in vivo. This has led to their application in a number of gene and cell therapies, particularly in circumstances where transgene capacity precludes use of AAV vectors, or cell targeting is suboptimal with non-viral vector technologies. Additionally, their ability to permanently integrate their DNA into dividing and non-dividing cells has made them a valuable tool in stem cell therapies, as modified cells will retain the therapeutic payload throughout cell division.

Here, the inventors show that HIV-1-based LV vectors can be used as transient mRNA delivery vehicles by engineering the reverse-transcriptase and RNA genome to promote translation of the transgene from the 5′ m⁷G cap, which they show delivers immediate, but transient mRNA expression both in vitro and in vivo.

The findings of their work are of relevance to retrovirology and the mechanism of HIV-1 uncoating. The precise mechanism of lentivirus uncoating and the timing of genomic RNA release is not well defined. A number of mechanisms have been proposed, one of which is that capsid disassembly occurs during the early stages of reverse-transcription. Given that reverse-transcription is dysfunctional in the inventors' system, but EGFP is clearly detectable soon after cell entry, this suggests that uncoating may not be absolutely dependent on reverse-transcription and a significant amount of vector RNA is released immediately after cell entry, irrespective of reverse transcription.

During the development of the CDLV system, the inventors engineered a variety of gene expression cassettes designed to modify the packaging and expression of vector ssRNA. Version 2 was taken forwards to further investigations, given that it produced the most efficient EGFP expression. The inventors demonstrated that this structure clearly maximizes in vitro expression compared to an IRES-based version, in a conventional 3^{rd} generation LV backbone. Further to this, the inventors also demonstrated that CDLV can transduce cells in vitro with comparable frequency to non-integrating and integrating 3^{rd} generation LV vectors, but with complete transiency. Additionally, direct comparison of CDLV to an integration-deficient LV (IDLV) vector showed that CDLV could achieve greater transduction frequency than IDLV, albeit with weaker expression levels per transduced cell.

Finally, given efficient liver targeting of VSVg-pseudotyped LV vectors the inventors investigated the ability of CDLV to deliver transient gene expression to hepatocytes in vivo, employing a luciferase reporter to track live vector expression kinetics in vivo. This study showed that CDLV could indeed provide short-term transgene expression in vivo, demonstrating a similar profile to that obtained in vitro. Luciferase expression from the CDLV platform was comparable to IPLV and IDLV 3^{rd} generation vectors during the early stages after injection, despite the 3^{rd} generation vectors being delivered at higher doses. Additionally, it is important to note that in all of experiments 3^{rd} generation IPLV and IDLV vectors were driven by a strong SFFV promoter, which will produce high levels of mRNA in hepatocytes and HEK 293T cells. In gene therapy, promoters weaker than SFFV are usually preferred, particularly in a lentiviral context, due to potential safety concerns. Therefore, the gene expression profiles that the inventors have detected from CDLV technology is likely to be comparable to clinically relevant lentiviral vector cassettes. However, validation of the in vivo scalability of their platform will require further studies beyond neonatal mice, given that larger animal models may not be easily transduced in vivo with lentiviral vectors.

The ability to deliver LV genomes as mRNA in vitro and in vivo brings some potential benefits in gene and cell therapy. LV vectors have a relatively large packaging capacity, able to package the mRNA of the majority of human genes. Therefore, CDLV technology has the potential to express large therapeutic transcripts transiently with high efficiency. This presents an advantage over non-viral gene transfer technologies, as transfection efficiency is known to reduce in correlation with increasing nucleic acid length. Indeed, LV vector gene transfer efficacy is also known to reduce in correlation with payload size, but it is noteworthy that this effect is thought to be limited primarily by inefficient reverse-transcription of large payloads, rather than ssRNA packaging, which suggests that CDLV payload tolerance could be even higher than that of conventional LV vectors.

Perhaps one of the most interesting avenues for exploiting CDLV technology would be delivery of genome editing nucleases. Cas9 nuclease mRNA has been used for in vitro and in vivo applications, although novel mRNA delivery strategies are continually being explored to enhance gene transfer efficiency. Lentiviral delivery of mRNA holds significant advantages here, as it has been shown over the past 20 years that LV vectors can be pseudotyped with a range of glycoproteins for targeted transduction of a wide range of cell types. Additionally, the packaging capacity of LV vectors means that Cas9 expression transcripts can be easily modified to include any of the recently developed modules (e.g. base editors, transcriptional activators and repressors), without compromising packaging ability.

An additional platform that could benefit from CDLV technology is in vaccinology, where researchers are developing methods to express antigens in antigen presenting cells (APCs) in situ. Lentiviral vectors have been investigated extensively for this purpose and it has been shown that a lack of pre-existing immunity allows repeated administrations. However, a potential limitation of LV use for APC transduction is the expression of SAMHD1 (SAM and HD domain-containing deoxynucleoside triphosphate triphosphohydrolase 1) in these immune cells, which lowers intracellular dNTP pools and reduces transduction efficiency by restricting the activity of reverse-transcriptase. Therefore, given that CDLV technology is not dependent on reverse-transcription for mediating expression, the inventors' platform technology could provide an advantage in this area of gene therapy.

In summary, the inventors report design and development of a novel LV gene structure that enhances translation of packaged genomic mRNA immediately upon cell entry, with limited duration. The inventors have shown that this novel CDLV vector can be used for gene expression both in vitro and in vivo, for potential applications in gene therapy.

Materials and Methods

Generation of Plasmid Constructs

All plasmid constructs were made using standard molecular cloning procedures and PCR-mediated deletion of plasmid sequences (Hansson, M D et al. (2008), Anal. Biochem. 375: 373-5). In cases where novel sequences or HIV-1 sequence deletions were incorporated, synthetic DNA fragments were designed and ordered as gBlocks (Integrated DNA Technologies).

Production of Lentiviral Vectors

Lentiviral vectors were produced as described previously (Vink, C A et al. (2017), Mol. Ther. 9: 10-20). Briefly, 1.8×10 7 HEK293T cells were plated per 15 cm sterile culture dish and transfected with the following components: 40 μg of the relevant transfer plasmid, 20 μg of pMDLg.RRE, 10 μg of pRS V-Rev and 10 μg of pMDG.2 (all plasmids produced by PlasmidFactory). Additionally, 10 μg of pCMV-Tat (kindly provided by Professor Axel Schambach from Hannover Medical School (Huelsmann, P M et al. (2011), BMC Biotechnol. 11: 4) was supplemented for enhanced vector titres. The plasmid mixtures were added to 5 mL Opti-MEM and filtered through 220 nm sterile filter units. Filtered DNA was combined with 5 mL Opti-MEM (Life Tech/GE) containing 2 μM polyethylenimine (PEI, Sigma). The resulting 10 mL mixture was incubated at room temperature for 10 minutes before addition to HEK 293T cells. After 4 hours, the transfection mixture was replaced with fresh culture medium. Virus supernatant was collected at 48 hr and 72 hr post-transfection. After each harvest, the collected medium was filtered through a cellulose acetate membrane (0.45 mm pore). Lentivirus harvests were combined before concentration by ultracentrifugation. Briefly, viruses were placed in polyallomer centrifuge tubes (Beckman Coulter) and centrifuged for 2 hr at 90,000×g at 4° C. in a Sorvall Discovery 90SE Centrifuge. Following centrifugation, the supernatant was removed, and pellet recovered in 200 μL Opti-MEM.

Titration of Lentiviral Vectors

Vector titration by flow cytometry: 1×105 HEK293T cells were plated into each well of a 6-well plate and transduced with a dose-escalation of concentrated lentivirus. For IDLV and IPLV vectors, EGFP measurements were made at 72 hours post-transduction, whereas CDLV analysis was performed at 16 hours post-transduction. Titres were calculated based on cell populations in the range of 5-30% EGFP+, as described previously (Vink, C A et al. (2017), Mol. Ther. 9: 10-20). EGFP positive cells were identified as described below in ‘Detection of eGFP expression in transduced cells’.

Vector titration by p24 capsid antigen: A p24 ELISA kit (Clontech product 632200) was used to determine the LV vector capsid number, according to the kit manufacturer's calculations, where 1 ng p24 is equivalent to ˜1.25×107 lentiviral particles.

Vector titration by ssRNA genome copies: ssRNA genome copies were quantified using a qRT-PCR titration kit (Clontech product 631235). In brief, vector RNA was initially extracted from viral particles using spin columns and quantified by nanodrop. The vector RNA copy number was then calculated using an RT-qPCR assay targeting the HIV-1 RNA packaging sequence and extrapolating the absolute value from a standard curve of known vector genome copy numbers.

Detection of eGFP Expression in Transduced Cells

Unless stated otherwise, 100,000 cells were analyzed for EGFP expression in a BD FACSArray Bioanalyzer. During analysis, live cells were determined by gating forward-light-scatter versus side-scatter and isolating the relevant population. EGFP-positive cells were determined by plotting EGFP fluorescence (detected using a 530/30 nm bandpass filter) versus emission from the yellow channel (detected using a 575/26 band-pass filter) to compensate for auto-fluorescence. Non-transduced controls were used to gate background expression in each channel. All flow cytometry data were analyzed by FlowJo software version 9.3.1 (Tree Star).

Animal Procedures

For in vivo investigations, outbred CD1 mice (Charles River), were time mated to produce neonatal animals. At postnatal day 1, non-randomised neonates were subjected to brief hypothermic anaesthesia and intravenously injected with LV vectors via the superficial temporal vein. Experimental groups were blinded during the course of in vivo investigations. Experiments were carried out under United Kingdom Home Office regulations and approved by the ethical review committee of University College London.

Longitudinal Tracking of Vector Expression In Vivo

To monitor LTR1 bioluminescence in vivo, 40 μl of the relevant luciferase expression vector was administered intravenously to 1-day old neonatal CD-1 mice. Vector doses were based on ssRNA titration results. Doses were calculated as 1×10¹³ vg/ml for IPLV, 5×10¹² vg/ml for IDLV and 4×10¹¹ vg/ml for CDLV. Images and bioluminescence data were gathered continually for 10 days, as described previously (Buckley, S M K et al. (2015), Sci. Rep. 5: 11842), by intraperitoneal injection with firefly D-luciferin (150 mg/kg) and imaged after 5 minutes with a cooled charge-coupled device (CCD) camera (IVIS Lumina II, PerkinElmer). Detection of bioluminescence in the liver was performed using the auto region of interest (ROI) quantification function in Living Image 4.4 (PerkinElmer). Signal intensities were expressed as photons per second per centimeter 2 per steradian.

Statistical Analysis

All statistical analyses were carried out using Matlab 2015a. A Kruskal-Wallis test with Dunn's posthoc analysis was used to compare vector titres. Student's t-test was used to compare mean fluorescence intensities and EGFP values. In vitro experiments in cultured cells were performed in 4 experimental replicates. Mouse sample sizes were limited to 4 animals per experimental group for in vivo investigations.

Sequences

The sequences of the constructs depicted in FIG. 2a are provided in the associated sequence listing and the position of the various features of the constructs in the sequences are as follows:

CDLV1—SEQ ID NO: 1

Promoter: nt 1-605; Intron: 753-885; EGFP transgene: 942-1661; WPRE: 1677-2265; 3′ LTR (ΔU3-R-U5): 2352-2585; ΔGag: 2723-3061; RRE: 3232-3465; pA: 3939-4073.

CDLV2—SEQ ID NO: 2

Promoter: nt 1-605; EGFP transgene: 619-1338; WPRE: 1364-1932; 3′ LTR (ΔU3-R-U5): 2029-2262; ΔGag: 2400-2738; RRE: 2909-3142; pA: 3616-3750.

CDLV3—SEQ ID NO: 3

Promoter: nt 1-605; EGFP transgene: 619-1338; WPRE: 1364-1932; bGHpA: 1952-2176; 3′ LTR (ΔU3-R-U5): 2248-2481; ΔGag: 2619-2957; RRE: 3128-3361; pA: 3835-3969.

CDLV4—SEQ ID NO: 4

Promoter: nt 1-605; EGFP transgene: 619-1338; WPRE: 1354-1942; bGHpA: 1952-2176; Intron: 2206-2338; 3′ LTR (ΔU3-R-U5): 2453-2686; ΔGag: 2824-3162; RRE: 3333-2566; pA: 4040-4174.

CDLV5—SEQ ID NO: 5

Promoter: nt 1-605; EGFP transgene: 619-1338; 3′ LTR (ΔU3-R-U5): 1440-1673; ΔGag: 1811-2149; RRE: 2320-2553; WPRE: 3031-3619; pA: 3620-3754.

CDLV6—SEQ ID NO: 6

Promoter: nt 1-605; EGFP transgene: 619-1338; U5: 1355-1449; ΔGag: 1576-1914; RRE: 2085-2318; WPRE: 2796-3384; pA: 3385-3519.

1. A retroviral RNA vector comprising a 5′ cap, a transgene, a 3′ long terminal repeat (LTR) and an RNA packaging sequence,
wherein translation of the transgene is initiated at the 5′ end of the transgene in a cap-dependent manner,
wherein the 3′ LTR and the RNA packaging sequence are located 3′ of the transgene.

2. A retroviral RNA vector according to claim 1, wherein the vector does not comprise a 5′ LTR.

3. A retroviral RNA vector according to claim 1, wherein the RNA packaging sequence comprises the RNA packaging signal (Ψ) and a portion of the gag gene.

4. A retroviral RNA vector according to claim 3, wherein the RNA packaging sequence also comprises the Rev Response Element (RRE).

5. A retroviral RNA vector according to claim 1, wherein the RNA packaging sequence is located 3′ of the 3′ LTR.

6. A retroviral RNA vector according to claim 1, wherein the vector does not comprise a primer binding site (PBS).

7. A retroviral RNA vector according to claim 1, wherein the vector further comprises an intron, optionally wherein the intron is located between the 5′ cap and the transgene.

8. A retroviral RNA vector according to claim 1, wherein the transgene further comprises a Kozak sequence.

9. A retroviral RNA vector according to claim 1, wherein the vector further comprises a post-transcriptional regulatory element (PRE), such as a woodchuck hepatitis virus post-transcriptional regulatory element (WPRE).

10. A retroviral RNA vector according to claim 1, wherein the vector further comprises a nuclear export signal, such as the constitutive transport element (CTE).

11. A retroviral RNA vector according to claim 1, wherein the 3′ LTR is a self-inactivating (SIN) LTR.

12. A retroviral RNA vector according to claim 1, wherein the vector is a lentiviral vector.

13. A retroviral RNA vector according to claim 12, wherein the vector is an HIV-1 vector.

14. A retroviral RNA vector according to claim 1, wherein the vector further comprises a poly(A) tail at the 3′ end of the vector.

15. A nucleotide sequence encoding a vector genome, the nucleotide sequence comprising a promoter operably linked to a vector genome,
wherein the vector genome comprises a transgene, a 3′ LTR and an RNA packaging sequence,
wherein the transgene is located at the 5′ end of the vector genome such that, following transcription and capping of the vector genome, translation is initiated at the 5′ end of the transgene in a cap-dependent manner,
wherein the 3′ LTR and the RNA packaging sequence are located 3′ of the transgene.

16. A nucleotide sequence according to claim 15, the nucleotide sequence further comprising a poly(A) signal.

17. A host cell containing:
(a) retroviral RNA vector comprising a 5′ cap, a transgene, a 3′ long terminal repeat (LTR) and an RNA packaging sequence, wherein translation of the transgene is initiated at the 5′ end of the transgene in a cap-dependent manner, wherein the 3′ LTR and the RNA packaging sequence are located 3′ of the transgene or
(b) a nucleotide sequence encoding a vector genome, the nucleotide sequence comprising a promoter operably linked to a vector genome, wherein the vector genome comprises a transgene, a 3′ LTR and an RNA packaging sequence, wherein the transgene is located at the 5′ end of the vector genome such that, following transcription and capping of the vector genome, translation is initiated at the 5′ end of the transgene in a cap-dependent manner, wherein the 3′ LTR and the RNA packaging sequence are located 3′ of the transgene.

18. A virion containing the retroviral vector according to claim 1.

19. A pharmaceutical composition comprising the retroviral vector according to claim 1 or a virion encoding the retroviral vector.

20. (canceled)

21. (canceled)

22. A method of delivering a transgene to a target cell, the method comprising administering an effective amount of the retroviral vector according to claim 1 or a virion encoding the retroviral vector to the target cell.

23. A method of delivering a transgene to a target cell in a subject, the method comprising administering an effective amount of the retroviral vector of claim 1 or a virion encoding the retroviral vector to the subject.

24. A cell produced by a method comprising administering to a subject an effective amount of:
(a) a retroviral vector comprising a 5′ cap, a transgene, a 3′ long terminal repeat (LTR) and an RNA packaging sequence, wherein translation of the transgene is initiated at the 5′ end of the transgene in a cap-dependent manner, wherein the 3′ LTR and the RNA packaging sequence are located 3′ of the transgene; or
(b) a virion encoding the retroviral vector.
